(19) United States
(12) Patent Application Publication
(10) Pub. No.: US 2025/0099301 A1
(43) Pub. Date: Mar. 27, 2025

(54) DEVICE FOR APPLYING PRESSURE TO STOP BLEEDING AFTER ARTERIAL AND VEINOUS PHLEBOTOMY PUNCTURES AND TO STOP BLEEDING AFTER A PUNCTURE WOUND

(71) Applicant: Bonnie Haragos, Escondido, CA (US)

(72) Inventor: Bonnie Haragos, Escondido, CA (US)

(21) Appl. No.: 18/371,433

(22) Filed: Sep. 21, 2023

(51) Int. Cl. A61F 13/00; A61B 17/12; A61B 17/132

(52) U.S. Cl. CPC A61F 13/00051; A61B 17/1325; A61B 2017/12004; A61F 2013/0028

(57) ABSTRACT

A method for applying pressure after an arterial or veinous blood draw or puncture wound so as to achieve hemostasis. First a gauze is placed over the wound. Then a dome is placed over the gauze. The dome has a rounded top and the rounded top is adjacent to the gauze and over the wound. Finally, a bandage is placed over the dome and tightened so that pressure is applied downward onto the wound to enable hemostasis. In one preferred embodiment, the present invention is used in medical setting, such as a hospital or medical office. In another preferred embodiment, in the event of an emergency the present invention may be used to apply pressure to a wound that occurs as the result of injury to achieve hemostasis.

The present invention relates to medical devices, and in particular, to devices for achieving hemostasis.

### BACKGROUND OF THE INVENTION

Phlebotomy is when a needle is used to take blood from a vein or artery, usually in the patient's arm. Also called a blood draw or venipuncture, it's an important tool for diagnosing many medical conditions and treating medical conditions.

Hemostasis is the stoppage of the flow of blood. Hemostasis includes three steps that occur in a rapid sequence: (1) vascular spasm, or vasoconstriction, a brief and intense contraction of blood vessels; (2) formation of a platelet plug; and (3) blood clotting or coagulation, which reinforces the platelet plug with fibrin mesh that acts as a glue to hold the clot together. Once blood flow has ceased, tissue repair can begin.

Whether a patient is bleeding due to a puncture wound or bleeding as a result of a needle prick, it is important to achieve hemostasis and stop the bleeding as quickly as possible. The inability to achieve effective hemostasis is a problem effecting many people. For example, it is common for dialysis patients to have difficulty achieving hemostasis after the needle is removed during dialysis. Typically, after dialysis is completed and the needle is removed, a patient is advised to place gauze and bandage over the needle hole and apply pressure with his or her fingers or thumb for approximately ten minutes. Alternatively, a fistula pressure clamp may be placed over the needle prick to apply pressure to stop bleeding. Unfortunately, both prior are methods are largely ineffective. The bleeding commences again as the patient leaves the medical office or hospital so that the patient returns home to discover that the bleeding has continued and bandages and clothing soaked in blood.

What is needed is a better device and method for achieving hemostasis after a wound or needle prick.

### SUMMARY OF THE INVENTION

The present invention provides a method for applying pressure after an arterial or veinous blood draw or puncture wound so as to achieve hemostasis. First a gauze is placed over the wound. Then a dome is placed over the gauze. The dome has a rounded top and the rounded top is adjacent to the gauze and over the wound. Finally, a bandage is placed over the dome and tightened so that pressure is applied downward onto the wound to enable hemostasis. In one preferred embodiment, the present invention is used in medical setting, such as a hospital or medical office. In another preferred embodiment, in the event of an emergency the present invention is used to apply pressure to a wound that occurs as the result of injury to achieve hemostasis.

### BRIEF DESCRIPTION OF THE DRAWINGS

FIG. 1 shows a preferred dome.

FIG. 2 shows another preferred dome.

FIGS. 3-14 show variety of preferred domes.

FIGS. 15=19 show a preferred embodiment of the present invention.

FIGS. 20-24 show another preferred embodiment of the present invention.

FIGS. 25-27 show another preferred embodiment of the present invention.

FIG. 28 shows a preferred kit.

FIG. 29 shows a preferred kit.

FIG. 30 shows a preferred dome with an keychain hole to facilitate attachment to a keychain.

### DETAILED DESCRIPTION OF THE PREFERRED EMBODIMENTS

The present invention provides a device that can be used to stop arterial and venous bleeding that occurs during phlebotomy. This device left on for more than 5 minutes will secure and stop the bleeding. This device may be used in a laboratory setting, medical office or hospital. In a home setting, the device may also be used to stop arterial, venous and capillary bleeding that occurs as the result of injury. The device may be provided in a sterile package. For example, a preferred sterile package might measure 4-inch×4-inch and include a sterile 2-inch×2-inch gauze, a dome and bandaging tape. Also, a non-sterile embodiment could include a dome that is readily available to the user. For example, the user might carry the dome attached to his key chain or the user might have the dome in the home's medicine cabinet. The non-sterile dome could then be used in conjunction with gauze and bandage to apply pressure to a wound in a fashion similar to that described below.

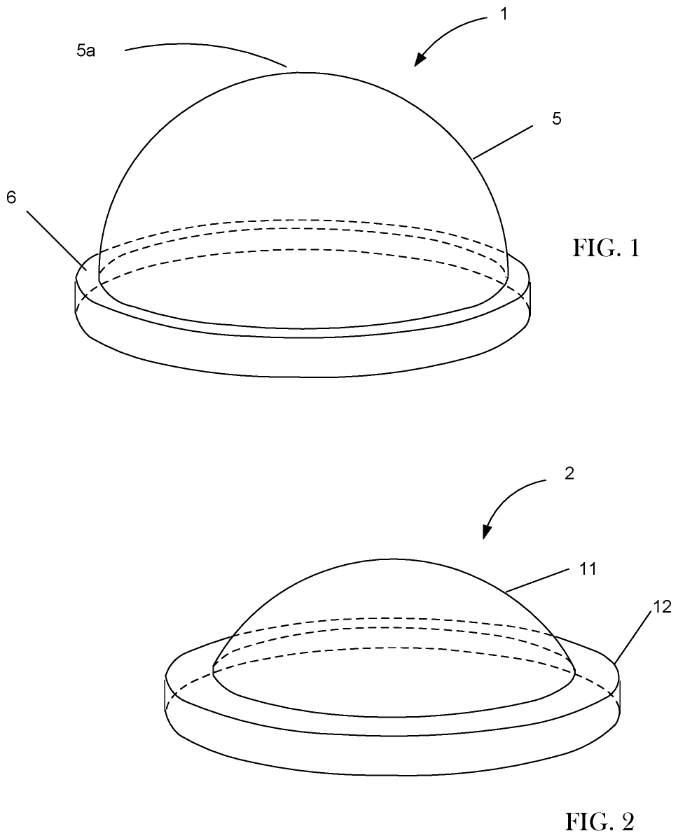

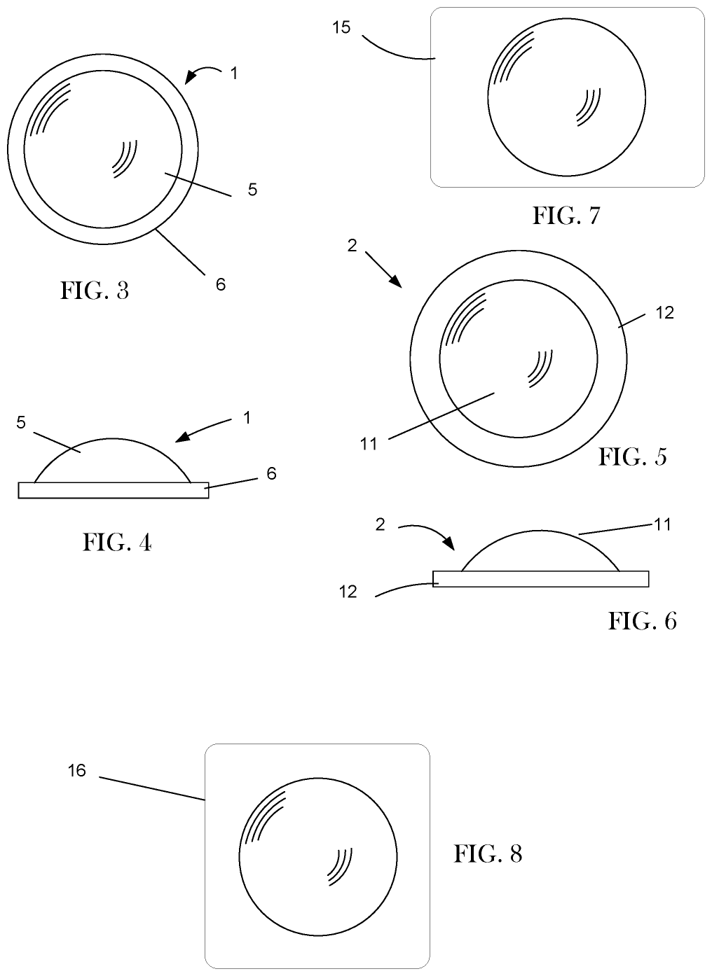

FIG. 1 shows preferred dome 1 and FIG. 2 shows preferred dome 2. Domes 1 and 2 are both preferably fabricated from non-toxic solid hard plastic. Dome 1 includes dome section 5 and base section 6. Dome section 1 includes rounded top 5a. Dome 2 includes dome section 11 and base section 12. The dimensions of the dome sections and the base sections may vary depending on the preferences of the user. For example, in one preferred embodiment, base section 6 has a diameter of 1 inch and a height of ⅛ inch. Likewise, dome section 5 may have a height of 0.5 inch and dome section 11 may have a height of 0.25 inch. FIG. 3 shows a top view and FIG. 4 shows a side view of dome 1. FIG. 5 shows a top view and FIG. 6 shows a side view of dome 2.

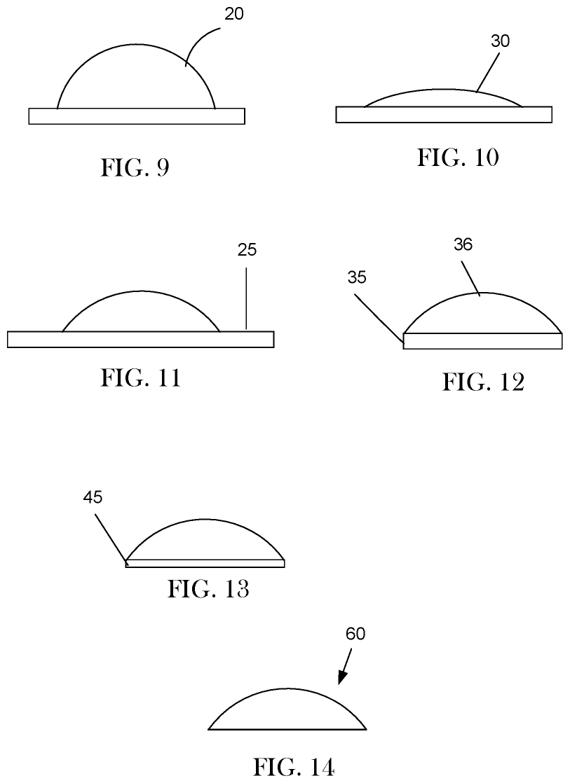

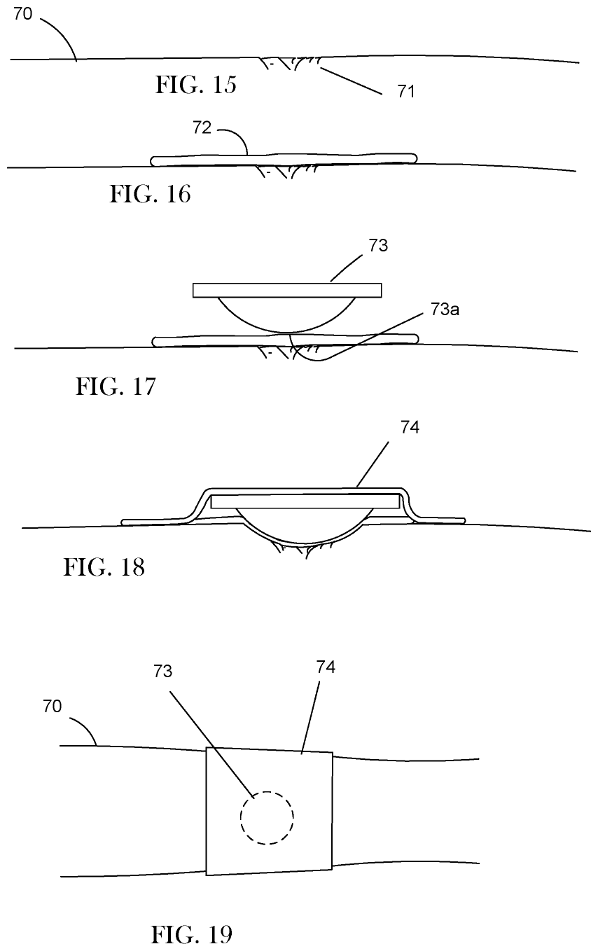

FIG. 7 shows that base section 15 is rectangular and FIG. 8 shows that base section 16 is square. The shape of the base may be modified to achieve different stability and pressure results. FIGS. 9-14 show that it is possible to modify the height of the dome section and the size of the base section to achieve preferred results. For example, FIG. 9 shows dome section 20 that is approximately ½ inch high. FIG. 10 shows dome section 30 that ⅛ inch high. FIG. 11 shows base section 25 that is approximately 2 inches in diameter. FIG. 12 shows base section 35 that is equal in diameter to dome section 36. FIG. 13 shows base section 45 that has a lower height of approximately 1/16 inch. FIG. 14 shows a dome 60 with no base section.

FIGS. 15-19 illustrate a method for using a preferred embodiment of the present invention.

In FIG. 15, a patient has wounded his arm 70 at wound 71.

In FIG. 16, to begin treating the wound the patient has laid sterile gauze 72 over the wound.

In FIG. 17, the patient has placed dome 73 over gauze 72 so that rounded top 73a is poised to apply pressure onto wound 71.

In FIG. 18 the user has wrapped bandage 74 tightly around his arm so that dome 73 extends downward into wound 71, thereby applying pressure to the wound.

FIG. 19 shows a top view of bandage 74 wrapped tightly around the users arm so that dome 73 exerts steady pressure to the wound as explained above in reference to FIG. 18.

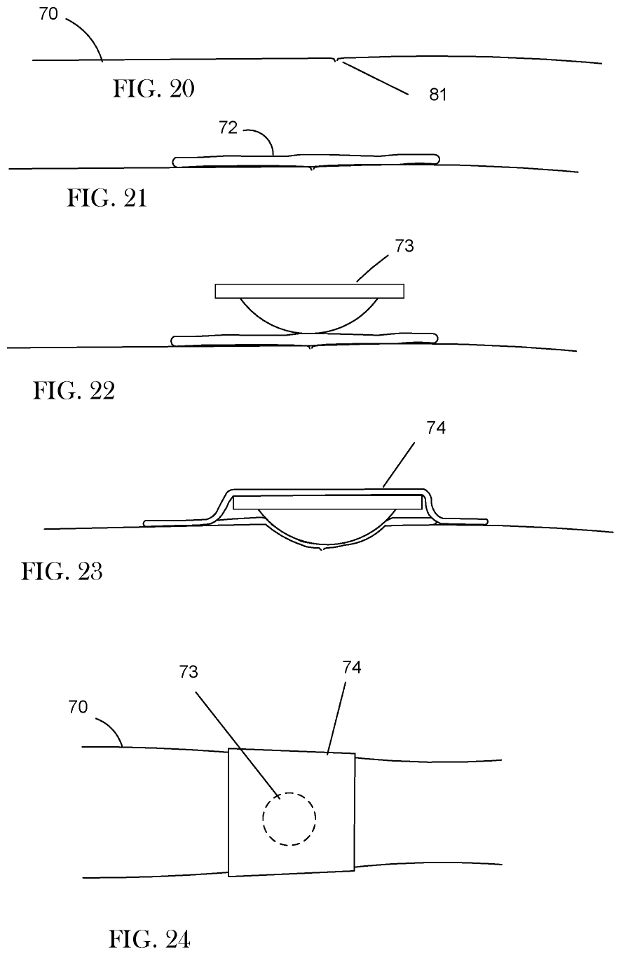

FIGS. 20-24 illustrate a method for using a preferred embodiment of the present invention to apply pressure to a needle prick.

In FIG. 20, a patient has removed a needle from his arm 70 after undergoing dialysis leaving the wound of needle hole 81.

In FIG. 21, to begin treating the wound the patient has laid sterile gauze 72 over the needle hole.

In FIG. 22, the patient has placed dome 73 over gauze 72 so that dome 73 is poised to apply pressure onto needle hole 81.

In FIG. 23 the user has wrapped bandage 74 tightly around his arm so that dome 73 extends downward into needle hole 81, thereby applying pressure to the wound.

FIG. 24 shows a top view of bandage 74 wrapped tightly around the users arm so that dome 73 exerts steady pressure to the wound as explained above in reference to FIG. 18.

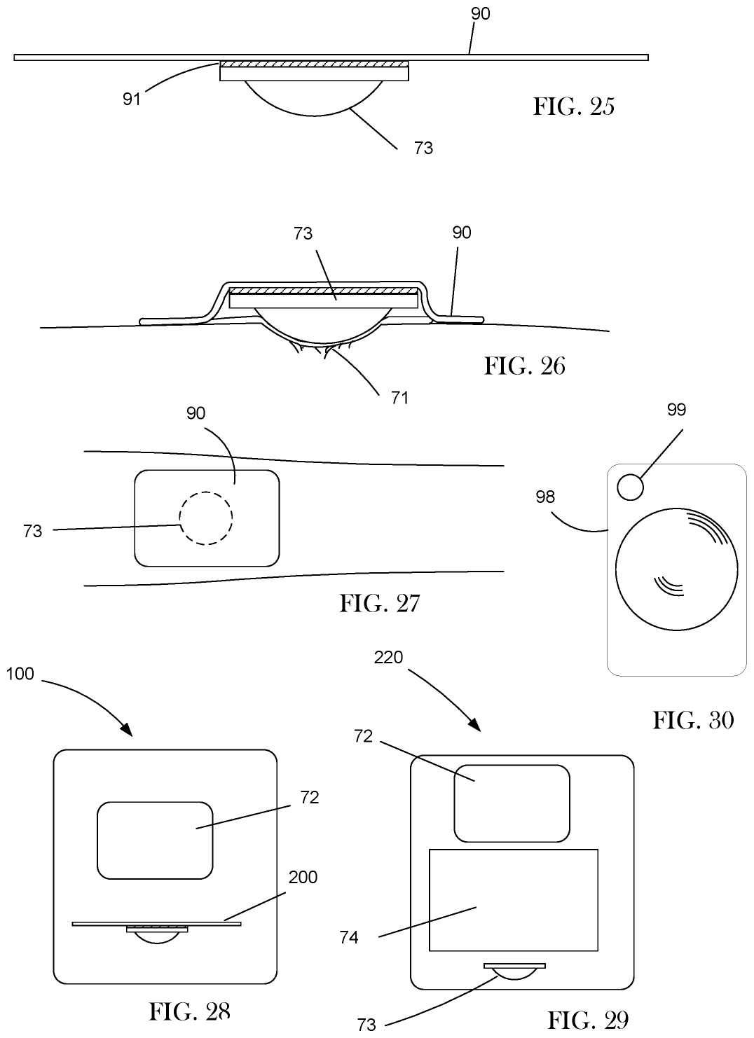

FIGS. 25-27 show another preferred embodiment of the present invention.

In FIG. 25, dome-bandage combined unit 200 includes adhesive section 91 connecting dome 73 to bandage 90.

In FIG. 26, the user has adhered bandage 90 to his arm to cover wound 71, in a fashion similar to that described above in reference to earlier described embodiments.

FIG. 27 shows a top view of bandage 90 adhered tightly to the user's arm so that dome 73 exerts steady pressure to wound 71.

In preferred embodiments of the present invention, the components of the present invention are provided in a sterile kit for easy and safe usage. For example, FIG. 28 shows kit 100. Kit 100 includes gauze 72 and dome-bandage combined unit 200. Also, FIG. 29 shows kit 220. Kit 220 include gauze 72, bandage 74 and dome 73.

It should also be noted that the present invention may be attached to a keychain for efficiency in the event of an emergency. For example, FIG. 30 shows dome 98 with keychain hole 99. Utilizing key chain hole 99, a user may connect dome 98 to his keychain for quick and easy accessibility.

Although the above-preferred embodiments have been described with specificity, persons skilled in this art will recognize that many changes to the specific embodiments disclosed above could be made without departing from the spirit of the invention. For example, although it was stated above that domes 1 and 2 are preferably fabricated from non-toxic solid hard plastic, they may also be fabricated from non-toxic flexible plastic. Therefore, the attached claims and their legal equivalents should determine the scope of the invention.

What is claimed is:

1. A method for applying pressure to a wound so as to achieve hemostasis, said method comprising the steps of:
A. placing a gauze over said wound,
B. placing a dome comprising a rounded top on top of said gauze so that said rounded top is adjacent to said gauze and is over said wound, and
C. placing a bandage over said dome so that pressure is applied downward onto said wound.

2. The method as in claim 1, wherein said dome is fabricated from non-toxic hard plastic.

3. The method as in claim 1, wherein said dome is fabricated from non-toxic flexible plastic.

4. The method as in claim 1, wherein said dome comprises:
A. a dome section, and
B. a base section rigidly attached to said dome section.

5. The method as in claim 1, wherein said base section is connected to said bandage via adhesive.

6. The method as is claim 1, wherein said gauze, said bandage and said dome are housed within a sterile kit for easy and safe access.

7. The method as in claim 1, wherein said wound is a needle prick.

8. The method as in claim 3, wherein said dome further comprises a keychain hole for easy connection to a keychain.

9. A method for applying pressure to a needle prick so as to achieve hemostasis, said method comprising the steps of:
A. placing a gauze over said needle prick,
B. placing a dome comprising a rounded top on top of said gauze so that said rounded top is adjacent to said gauze and is over said needle prick, and
C. placing a bandage over said dome so that pressure is applied downward onto said needle prick.
